(19) United States
(12) Patent Application Publication
(10) Pub. No.: US 2022/0221336 A1
(43) Pub. Date: Jul. 14, 2022

(54) APPARATUS FOR CONTROLLING A PROCESS AND ACCOMPANYING CONTROL METHOD

(71) Applicant: BioThera Institut GmbH, Freiburg (DE)

(72) Inventors: Roland MERTELSMANN, Freiburg (DE); Joschka BÖDECKER, Freiburg (DE); Dennis RAITH, Stuttgart (DE); Jonas BERMEITINGER, Schliengen (DE)

(73) Assignee: BioThera Institut GmbH, Freiburg (DE)

(21) Appl. No.: 17/573,798

(22) Filed: Jan. 12, 2022

(30) Foreign Application Priority Data
Jan. 13, 2021 (DE) 102021100531.0

(51) Int. Cl. G01J 3/02; G01J 3/44; G01J 3/42

(52) U.S. Cl. CPC G01J 3/027; G01J 3/42; G01J 3/4406

(57) ABSTRACT

For the improved autonomous control of a process (2) using an apparatus (1) via setting of at least one action parameter (3) controlling the process (2), it is provided that via a measuring instrument (17), preferably a spectrometer (18) integrated into the apparatus (1), a process response (4), which the process (2) transfers in reaction to an adjustment of the at least one action parameter (3) to its immediate environment, is measured and evaluated in a computer-implemented manner, preferably using an artificial intelligence, and that based on this evaluation, the at least one action parameter (3) is automatically readjusted. This approach is applicable to biological, chemical and physical processes.

### INCORPORATION BY REFERENCE

The following documents are incorporated herein by reference as if fully set forth: German Patent Application No. 10 2021 100 531.0, filed Jan. 13, 2020.

### TECHNICAL FIELD

The invention relates to an apparatus for the autonomous control of a process. In particular, such a process can be a biological, or else also a chemical or physical process. In specified applications, the process can also be a conversion process, in which new products are obtained from starting materials or starting compounds, for example in the form of new substances or compounds or in the form of new physical objects such as particles of specified shape and/or size.

The invention further relates to an accompanying method for controlling an apparatus in which such a process takes place, wherein the process is controlled by setting at least one action parameter.

### BACKGROUND

Processes can in particular be carried out by a biological system. The biological system can be formed for example from cells and/or microorganisms and/or enzymes. The biological system can therefore comprise in particular a plurality of elements. The elements can for example be cells and/or microorganisms and/or enzymes. The biological system can be embedded in a culture medium and together therewith form a biological sample. For example, the culture medium can be a nutrient bed for a biological system composed of cells.

In particular, the biological process can be a growth or a metabolism or a biological function of a cell, wherein the cell for example can be cultivated in a culture medium inside a culturing chamber of the apparatus. The cell may be e.g. a cancer cell or a microorganism. The biological process can in particular be a microbiological process.

Alternatively, the process can also be a chemical or a purely physical process, such as for obtaining specified microscopic particles.

The basic characteristics of the methods and devices described above are already known. For example, methods are known by which the composition of a culture solution or other factors that influence the growth of cells of a biological sample is set by an apparatus for culturing the cells, wherein the apparatus has independently learned how these factors are to be set in order to allow optimum growth. However, the concrete selection of the process factors to be changed, such as a heating power for setting an overall temperature of the biological sample, and monitoring/regulation thereof by means of the apparatus, has been found in practice to be challenging and not always reproducible.

### SUMMARY

Based on this previously known prior art, the object of the invention is to allow improved, i.e. in particular more stable and more precise, control of processes with apparatuses such as those mentioned at the outset.

In order to achieve this object, one or more features according to the invention are provided in a method for controlling an apparatus in which a process such as that mentioned at the outset takes place. In particular, it is thus provided according to the invention, in order to achieve the object in a method of the type mentioned at the outset, that a process response of the process, which is returned by the process in reaction to the at least one action parameter, is measured, that this process response is evaluated by an evaluation in a computer-implemented manner using a predetermined preset target value, and that at least one set value of the at least one action parameter is adjusted in a computer-implemented manner based on the evaluation. In this case, the process response can thus be understood as a response of the (biological/chemical/physical) system that is responsible for/carries out said process.

In other words, in order to achieve the object mentioned at the outset in a method of the type mentioned at the outset, the invention therefore proposes acquisition of the manner in which the process reacts to an adjustment of at least one action parameter (with which the process is controllable). In this case, the reaction of the process (i.e. for example a biological system in particular that is formed by the cells and/or microorganisms involved) is recorded as a process or system response and is evaluated by means of an evaluation so that by means of the evaluation, a readjustment of the action parameter is carried out. This makes it possible to create a control loop that continuously and autonomously controls the process without any external intervention. For example, this method can be implemented using an apparatus according to the invention that will be described in further detail below.

In particular, the action parameter can be a process parameter with which an influence can be exerted on the process. This includes for example environmental conditions such as a temperature, a pressure, or a humidity. For example, an action parameter can also be provided by a volumetric flow of a substance that is supplied to the process.

The setting of action parameters can also include setting the action parameters at several points in time or in a temporally continuous and changeable manner.

It can be provided that several or a plurality of (particularly biological) processes take place in the apparatus in a parallel and/or serial manner. It can be advantageous if different action parameters are set for at least two processes running in parallel. In alternative experiments, the action parameters can also be set to be identical.

It can further be provided that during the course of a process, the process response thereof and/or the process status thereof is repeatedly acquired and/or evaluated. This is preferably carried out continuously.

The above method can be used in various application cases: in a first case, the method can be used to initially configure an apparatus such as that described at the outset, i.e. in order to determine configuration parameters for the at least one action parameter. Once the apparatus is configured, the apparatus is capable in a highly efficient manner of precisely controlling a (particularly biological) process using the determined configuration parameters, even if the process is slightly different from the process that was used for configuring the apparatus. In the first case, the method is thus used for initial configuration of the apparatus.

In the second case, however, the method is used for continuous control of the apparatus. In the second case, the apparatus can be used in a highly efficient manner and makes it possible, in particular via the control of a plurality of parallel, similar processes, to obtain a great deal of information on the respective process in an extremely brief period, which is of great interest in particular for the application to biological processes. In this manner, for example, the development of medicines can be greatly accelerated.

It is advantageous in both cases that without or with minimal personnel expense, a respective process can be carried out, preferably autonomously, by means of the apparatus and using the method according to the invention, in order to lead to a desired behavior. The desired behavior can exist with respect to a wide variety of parameters, for example a viability or suppression of cells or a growth/a proliferation of cells (in the case of a biological process), a desired material production, or a specified substance conversion (in the case of a chemical process, or (e.g. through metabolism of a substance to be degraded in cells or the production of a specified substance by the cells) or for example the formation of emulsion droplets with specified characteristics (in the case of a physical process).

By adjusting the action parameter, conditions, in particular environmental conditions or growth or nutritional conditions, can be altered or produced that influence the (particularly biological) process and thus control it. This allows the process to be guided in the direction of a desired system status. In the case of a biological process, which in particular is carried out by a biological system formed by cells, a specified reaction to these conditions, for example, can be output as a process or system response. This means that the process response is acquired in order to draw from it conclusions on a current status of the biological process or the respective system on which the process is based.

The adjustment carried out can lead to an optimization of the at least one action parameter with respect to an improved process response. For example, after adjustment/optimization of the at least one action parameter, the process response can approach a preset target value.

Such a preset target value, e.g. by means of pre-experiments, can be derived for example from a process status of a biological process. The respective process can be achieved in particular with the method or can be reproducibly set.

The preset target value can form an evaluation criterion for evaluation of the respective process response and/or the respective process status, or it can be provided that such an evaluation criterion is derived from the preset target value. The evaluation criterion can for example be a reward, cost, or penalizing function.

For example, an evaluation criterion can be derived from the aforementioned preset target value that provides that a deviation of the measured process response is evaluated by a reference response.

However, if the current status of the (particularly biological) process is to be evaluated, a cell density, for example of viable cancer cells, can be used as a status criterion.

It can also be provided that the preset target value evaluates a set parameter, such as the amount of a medication administered in a specified period of time.

For example, the preset target value can specify a weighted reward function. In particular, it can be provided that the preset target value evaluates a status in the categories of better or worse. It can be provided that a reward function is specified that can accept real numbers in a range, for example between 0 and 1, wherein a first real number that is closer to 1 than a second real number is evaluated better than the second real number.

It can also be provided that the preset target value provides a reward function that has only two statuses, for example 1 and 0 or infinity and 0 or the like. In particular, it can be provided that the preset target value evaluates a status in the categories good or bad. It can be provided that a reward function is to be maximized.

Equivalent to this, it can be provided that the reward function is a cost function that is to be minimized. For example, in this case a 0 cannot cause any costs and a 1 can cause high costs. A cost function can for example represent a number of time steps. This can be advantageous e.g. when a goal is to find a time-optimized solution, for example because a reference response is specified in a temporally changeable manner and such a response is to be achieved to the extent possible by the process response.

The acquisition of the process response can preferably take place in an automated manner, in particular in a computer-implemented manner, or else manually, for example at regular intervals.

The acquisition of the process response, its evaluation, the setting of the at least one action parameter, and all other measures discussed above can take place in a computer-implemented manner, preferably by means of an artificial intelligence (AI), which will be described in further detail below. This AI can also define the preset target value, in particular individual target variables of the preset target value.

The at least one action parameter can for example comprise an entire series of different action parameters. These can for example be combined in an action vector, the individual components of which are formed by the respective action parameters. Each action parameter can be used to set a respective regulating variable of the apparatus. In this way, the action vector can describe a specified current regulation status that is set in the apparatus in order to control the respective (particularly biological) process.

According to the invention, the object can also be achieved by further advantageous embodiments described below and in the claims.

For example, the evaluation of the process response can take place using a reference response for the process response. This reference response can then constitute the above-mentioned preset target value. In particular, this makes it possible for the process response to be aligned with the preset target value.

On the one hand, the preset target value can be determined in a deterministic manner, for example from a model of the process, in particular by means of a simulation. Alternatively, the preset target value can also be empirically determined, for example in that the process response of the process is recorded in a pre-experiment as a preset target value when the process is in a desired process status (such as optimum metabolic production or a desired cell death of cancer cells or a spectrum measured at this time).

The reference response can further comprise a plurality of target variables. In particular, the reference response can thus comprise a measured and/or a virtual, in particular simulated or calculated target variable. Virtual target variables can be provided in particular by the above-mentioned AI.

In a further embodiment, it can be provided that the process response of the (particularly biological) process is measured on a medium that surrounds elements of the system on which the process is based, in particular wherein the (for example biological) system is embedded in the medium forming a culture medium. In this manner, the surrounding medium is frequently changed by the process response. Measurement data on the medium for acquisition can therefore contribute toward an important gain in information for improving the control.

It can further be provided that the process takes place in a sample, in particular a microfluidic and/or biological sample. This sample can also comprise a culture medium. In this case, the process response can be measured in the culture medium. This is particularly suitable for investigating the growth or metabolism of microorganisms.

In turn, in other applications, for example in the control of a biological process for the production of artificial meat, the biological sample can be a macroscopic sample, which for example comprises a volume of several liters or even hectoliters. This means that the method presented here is particularly suitable for applications in the processing industry, particularly in the biotech area.

In both microscopic and macroscopic biological/chemical/physical samples, it is possible for the measurement of the process response to take place under exclusion from the system. For example, in a biological system, the measurement can take place under exclusion of the cells and/or microorganisms that are contained in the biological sample in which the process takes place. It can therefore be provided in particular that at least one measurement parameter of the process response is measured in an immediate environment of the elements of the (particularly biological) system, in particular in an immediate environment of cells and/or microorganisms, but specifically without direct measurement of the elements of the system.

By the measurement of the process response, a consumption and/or the production of at least one material by the (particularly biological) process can also be at least indirectly or directly acquired. This can preferably take place while the process is being influenced by adjusting the action parameter and/or wherein, by measurement of the process response, an activity of the system, in particular an activity of cells and/or microorganisms in the case of a biological system, is acquired. For example, such an activity can be a thermogenesis (production of heat by metabolic activity of the biological system) and/or a chemo- or biogenesis (production of chemical substances or biological organisms by metabolic activity) and/or a photogenesis (production of light by metabolic activity) and/or an energy consumption and/or a material consumption that is attributable to the process in each case.

The process, in particular the system on which the process is based, for example said microorganisms mentioned before, can also change at least one environmental factor, such as for example a material composition, for example of microorganisms or a culture medium surrounding them, and/or a temperature and/or a pH value and/or a permittivity and/or an electrical conductivity and/or an optical transmission, absorption or reflection behavior, to cite only a few examples.

In particular, these environmental factors can be factors of a culture medium in which a biological system is cultured. For this reason, it can be provided that at least one such environmental factor is measured as a measurement parameter of the process response. This makes it possible to acquire a change in the environmental factor in reaction to the adjustment of the at least one action parameter, which in turn allows conclusions to be drawn on the current status of the (particularly biological) process.

In a particularly advantageous embodiment, it is provided that at least one measurement parameter of the process response is acquired by means of a spectrometric measurement. This is applicable to biological, chemical, and physical processes.

In the case of a biological process, a culture medium, in which the biological system is cultured, can preferably be spectrometrically measured for this purpose. In order to always allow the process response to be consistently acquired in a particularly reproducible manner, it can be provided that only the culture medium, but not the cells and/or microorganisms contained therein, is illuminated. In other situations, however, it can be advantageous for this purpose to illuminate both the culture medium and the elements contained therein. This can take place for example by means of an OD-600 measurement, in which an optical density (OD), i.e. an absorption of the culture medium and the cells contained therein, is determined at a wavelength of 600 nm.

The spectrometric measurement can take place using illumination light in the UV and/or VIS and/or IR wavelength range. Moreover, a spectrum, in particular a transmission or absorption spectrum, can be acquired over a specified wavelength range as a measurement parameter/as a process response. In this case, a target spectrum can be used as a preset target value. For example, the target spectrum can be determined in a liquid, the composition of which is changed by the respective (chemical/biological/physical) process.

A preferred embodiment that is particularly advantageous in use of a microfluidic apparatus provides that the spectrometric measurement is carried out by means of a fiber optic unit. This fiber optic unit can be integrated into a microfluidic apparatus in which the process takes place. This is advantageous in that a sensor for recording the spectrum can be arranged outside the microfluidic apparatus, which results in a robust measurement solution.

As was already mentioned, the process and/or the system on which it is based can change a composition of a culture medium used in the apparatus. For this reason, the process response can also depend on the composition of the culture medium. However, this means that the process response can be measured by means of an investigation of the culture medium, preferably under exclusion of the (particularly biological) system. The change in the composition of the culture medium can for example result from the fact that the system consumes substances of the culture medium or releases metabolic products into the culture medium. This applies for both chemical and biological processes.

The process response, in particular measured measurement parameters, can be selected in such a way that it is possible to draw quantitative conclusions from the measured process response with respect to a material consumption and/or a material production by the process/the system.

The process response can in particular be acquired electrically and/or optically and/or inductively. It can further be provided that the acquisition of the process response comprises taking a (particularly biological/chemical/physical) sample, wherein the sample is investigated and the process response is derived from this.

Moreover, the acquisition of the process response can be carried out continuously or discontinuously.

Finally, the measurement of the process response itself can be temporally controlled based on the acquired process response. For example, in the case of a significant change in the process response, or when points of the process response are approached that are critical for the control of the process, a temporal frequency of the acquisition of the process response can be adjusted, for example in order to achieve a higher temporal resolution of the process response.

According to a further aspect of the invention, at least the adjustment of the respective set value of the at least one action parameter, preferably and the preceding evaluation of the measured process response, can take place by means of an artificial intelligence (AI) in a computer-implemented manner. Such an AI can be realized for example by means of a method of machine learning, preferably by means of a method of deep learning and/or using artificial neural networks (ANN). The ANNs may comprise numerous non-accessible hidden layers.

The artificial intelligence can in particular be configured in a model-free manner. Model-free means that no model of the process or the control of the process is specified; rather, the AI learns the control of the system/the process solely from observations.

In this manner, at least one optimized set value for the at least one action parameter can be independently learned by the apparatus in a computer-implemented manner based on several evaluations derived by the apparatus from a respective process response (in each case in reaction to set values of the at least one action parameter specified by the apparatus). Using such set values of the at least one action parameter (i.e. for example using a volumetric flow to be set as a possible action parameter), a respective control variable of the apparatus (i.e. for example a valve setting that defines the volumetric flow) can be adjusted. As is the case for all other adjustments, this adjustment can also preferably be carried out in a computer-implemented manner by the apparatus itself (i.e. autonomously from human interventions).

According to a further possible embodiment, in particular within the framework of a design of experiment (DoE), it is provided that the above-mentioned artificial intelligence (AI) first creates data sets that comprise a respective measured process response and an accompanying set of action parameter values, wherein it is precisely these action parameter values that generate the process response. The AI can then use these data sets to prepare and/or optimize a prediction model for the system response. This prediction model thus describes how the process reacts to specified set action parameters. In this case, the prediction model need not be an explicit model, which would require exact knowledge of the process taking place in the sample/the system. Rather, in the simplest case, the prediction model can be characterized by weightings, for example in an artificial neural network, by means of which the AI predicts the process response in reaction to a specified set of action parameter values. Such a prediction model constitutes an additional output that can be generated by means of the method. It is obvious that such an output can be valuable in order to optimize the control of biological, chemical or else physical processes.

A particularly preferred embodiment further continues this approach in that the AI, with the help of the prediction model, generates virtual system responses in reaction to respective virtual sets of action parameter values. This means that the AI virtually calculates system responses based on the prediction model obtained from the measurement data of the process response that can be expected for a specified set of action parameter values. The calculated virtual system responses can then be classified by the AI using the preset target value. In this manner, such virtual system responses can be selected that are of interest, i.e. such system responses that indicate that the process takes place in the direction of the preset target value or already in a desired target state.

Finally, the AI can then validate the virtual system responses, particularly those that are selected, by means of real tests. For this purpose, the AI carries out tests with the help of the apparatus in that the AI controls the apparatus with a respective virtual set of action parameters and measures the real process response arising therefrom. The AI can then subsequently compare the real process response with the respective virtual system response that was previously calculated by the AI based on the prediction model.

With this approach, it is thus possible to identify virtual system responses that deviate significantly from the real behavior of the process. For this reason, the AI can in turn further optimize the prediction model by means of the validation carried out. It is obvious that this entire optimization process can be iteratively repeated. A substantial advantage of this approach is that the AI carries out only such experiments for which it can be assumed with a high degree of probability that they will be valuable for optimizing the control of the apparatus. In this manner, meaningless experiments are avoided, which substantially shortens the time required for the optimization of the control of the process, in particular when a large number of action parameters is to be set and/or when the process is highly complex.

It can also be provided that in addition to the process response, a process status is measured. Preferably, a first measurement parameter is measured by the process response, and a second measurement parameter is measured by the process status. This allows an additional information gain to be achieved, by means of which the control can be improved.

It can further be provided that at least two measurement parameters are measured, and that one of the two measurement parameters is used in order to verify an evaluation carried out by means of the other measurement parameter. Preferably, the evaluation is changed depending on a result of the verification, and particularly preferably by an AI, in particular the above-mentioned AI.

Alternatively, measurement results and/or measurement parameters can also be evaluated independently of one another and taken into account by the AI for control of the process.

The process response can in particular comprise at least two different measurement parameters. For example, at least two measurement parameters that both characterize the process response can therefore be measured.

However, additional measurement parameters that pertain to the status of the process can also be measured. In particular, a first measurement value can pertain to the process status and a second measurement value can pertain to the process response.

For robust control, it can be advantageous if the measurement parameters that characterize the process response are selected from the following group of measurement parameters: optical measurement variables, such as an absorption spectrum, an emitted light intensity, or a fluorescence; electrical variables, in particular an electrical conductivity and/or permeability and/or capacitance; thermal variables, in particular a self-heating of a chemical or biological sample in which the process takes place; pH values.

The process response and/or the process status, in particular individual measurement parameters, can thus be acquired optically and/or electrically and/or inductively or for example by means of a pH sensor.

Using a respective preset target value (or a respective target variable), each of the measurement parameters can be characterized or evaluated by means of a respective evaluation in a computer-implemented manner. At least one set value of the at least one action parameter can be adjusted in a computer-implemented manner here based on the totality of these evaluations or a selection of these evaluations. Preferably, an artificial intelligence can then use the evaluations as an input vector, in particular such that all of these evaluations are taken into account in determining the set value.

It can further be provided that a first measurement parameter is used to verify and/or adjust an evaluation criterion which is used to generate an evaluation of a second measurement parameter. In such a case, it is advantageous if this takes place by means of a method of self-supervised learning in which the first measurement parameter is taken as a basic truth in order to improve the evaluation of the second measurement parameter.

For example, an absorption spectrum as a first measurement parameter can be used to adjust an evaluation criterion that is used in optical image analysis of recorded image data, for example from cell cultures. These image data can then be characterized as a second measurement parameter using the adjusted evaluation criterion by means of an evaluation. It is also conceivable to reverse this procedure.

In other words, an artificial intelligence can learn, based on a first acquired measurement parameter, how a second measurement parameter is to be correctly evaluated in order to carry out an appropriate adjustment of a set value of the at least one action parameter based on this learned and thus improved evaluation. The AI thus learns the correct evaluation of a second measurement parameter based on the acquisition of a first measurement parameter (which is used as a label). For example, acquired spectra can be used in order to label/classify training data in the form of accompanying microscopic images in order in this manner to learn correct image recognition of healthy organisms in the microscopic images.

This makes it possible to dispense with manual classification of the microscopic images.

At least one of the acquired measurement parameters can be an overall measurement parameter that is influenceable by all of the microorganisms. Examples include a temperature increase in a biological sample in which the biological process takes place or a transmission spectrum of a culture medium that nourishes the microorganisms and into which these microorganisms release metabolic products. This overall measurement parameter can in particular be acquired without direct measurement of the microorganisms, for example through a spectrometric measurement of a culture medium, as discussed above.

It can also be provided that at least one of the acquired measurement parameters is a local measurement parameter that is only influenceable by individual organisms. For example, such a local measurement parameter can be a specified number of healthy cells in a specified measuring range or an average size of a selection of cells. The local measurement parameter can thus in particular be acquired by direct measurement of individual microorganisms, for example by means of an image processing of microscopic images of the microorganisms.

In order to achieve the above-mentioned object, the features of the independent device claim are further provided according to the invention. In particular, in order to achieve the object in an apparatus of the type described at the outset, it is therefore proposed according to the invention that the apparatus comprise the following components: a process chamber for accommodating a biological, chemical or physical system in which a process (particularly a biological, chemical or physical process as described at the outset) takes place; a controller configured to adjust at least one action parameter in order to control the process by means of the action parameter; and at least one measuring means for measuring a process response of the process that takes place in reaction to a setting and/or adjustment of the at least one action parameter.

Here, the at least one measuring means can in particular be a spectrometer and/or a camera. By means of this embodiment, methods according to the invention as described above or according to one of the claims pertaining to a method can be simply implemented with the apparatus. Such an apparatus is suitable for controlling biological, chemical and physical processes.

The controller can further be specifically configured to carry out a method according to the invention as described above or according to one of the claims pertaining to a method.

If the apparatus is to be used for the control of a biological process, the process chamber can be configured as a culturing chamber that comprises a culture medium for culturing a biological system, i.e. for example cells and/or microorganisms. In this case, it can further preferably be provided that at least one of the measuring means is configured to acquire an overall measurement parameter of the process response, which is changeable or is changed by all of the elements of the biological system, in particular by all of the cells and/or microorganisms of the system.

Additionally or alternatively, it can also be provided that at least one of the measuring means, in particular a measuring means configured for acquisition of an, in particular the above-described, overall measurement parameter, is arranged such that the measurement of the process response takes place at least partially at a measuring point that is kept free from the biological system, in particular from the cells and/or microorganisms. This allows a change in the environmental conditions that are attributable to metabolic processes or other changes in the biological process to be reproducibly detected (and used for adjustment of the control).

Preferably, it can further be provided that the measuring means is designed, arranged and/or configured such that a measurement value is acquired that is determined by an environment of elements of the biological system. For example, the environment can be the culture medium in which the biological system is cultured.

A further preferred embodiment, which is suitable in particular for controlling cells and/or micro-biological processes but also chemical or physical processes, provides that the apparatus comprises a microfluidic device for supplying the system with a medium, in particular a liquid or the above-described culture medium. For example, this device can comprise a plurality of clamps or containers in each of which biological samples can be kept. Furthermore, the device can also be configured as a flow-through device so that the culture medium can flow in the form of a liquid past the elements of the biological system.

It can further be provided that the device comprises a microfluidically active separating structure with which the biological system, in particular the microorganisms, can be kept distant from a, particularly the above-described, measuring point, at which the process response can be acquired with the at least one measuring means. Examples of possible separating structures are a membrane, a filter, or microscopically dimensioned through-flow openings by means of which the microorganisms can be held back.

Finally, the at least one measuring means can at least partially be integrated into the microfluidic device. In this case, the measuring point can thus be unchangeable with respect to the separating structure, which leads to a reproducible and precise acquisition of the process response. Even when a separating structure is dispensed with, the integration can be advisable in order to increase the robustness of the measurement.

The apparatus can further comprise at least one actuator, such as a controllable valve, with which the at least one action parameter is changeable and wherein the at least one actuator is controllable by means of the controller.

It can further be provided that the controller is configured to adjust at least one set value of the at least one action parameter in a computer-implemented manner based on an evaluation of the process response, specifically in particular by comparison with a preset target value for the process response. In this manner, for example, the controller can bring the process response to the preset target value.

Finally, it should be mentioned that the apparatus can of course be configured for carrying out one of the methods discussed above and/or can comprise the means necessary for carrying out one of the methods discussed above (measuring means, regulating means, electronic memories, sampling device, etc.).

The invention will now be described in further detail by means of exemplary embodiments, but is not limited to these exemplary embodiments. Further embodiments of the invention can be derived from the following description of a preferred exemplary embodiment in connection with the general description, the claims, and the drawings.

### BRIEF DESCRIPTION OF THE DRAWINGS

In the following description of various preferred embodiments of the invention, elements that are consistent in their function are given consistent reference numbers, even when they are of deviating configuration or form. The example presented relates to the control of a biological process. However, based on the explanations, the method according to the invention and the accompanying device can also be applied by a person skilled in the art to chemical or physical systems and processes.

The FIGURE shows the following:

FIG. 1 a schematic overview of an apparatus according to the invention for controlling a biological process that takes place in the apparatus.

### DETAILED DESCRIPTION

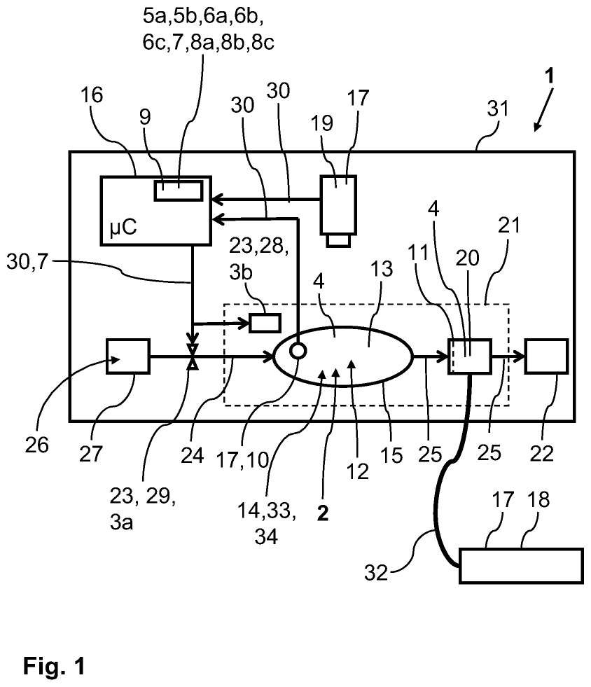

FIG. 1 shows an apparatus designated 1 as a whole, by means of which a biological process 2, which takes place in the apparatus 1, can be controlled autonomously, i.e. without external human intervention. The apparatus 1 comprises a culturing chamber 15 in the form of a microfluidic chamber through which a culture medium 13 can be guided that nourishes a biological system 34, which is cultured in the culturing chamber 15. The biological system 34 can consist in particular of elements 14 or comprise such elements, wherein the elements are cells 14 and/or microorganisms 14. The biological process 2 thus takes place in the microfluidic biological sample 12, which is formed by the biological system 34 and the culture medium 13 in the culturing chamber 15.

The apparatus 1 further comprises a reservoir 27 in which multiple substances 26 are stored, which can be fed via the supply line 24 shown (which comprises multiple separate lines) into the culturing chamber 15 in order to thus influence the biological process 2 taking place there as a respective action parameter 3a. Here, the actual action parameter 3a constitutes the flow rate at which a specified substance 26 is fed into the culturing chamber 15. This means that there are several action parameters 3a, each of which characterizes a flow rate of one of the substances 26 from the reservoir 27 to the culturing chamber 15.

In order to set this action parameter 3a, the apparatus 1 comprises at least one actuator 23 in the form of a valve 29 that is connected to a controller 16 via a data line 30. The controller 16 can thus access the adjustable valve 29 in a controlling manner and thus regulate or set the flow rate, i.e. the action parameter 3a.

The apparatus 1 further comprises a further actuator 23 in the form of a heating element 28 with which an overall temperature prevailing in the culturing chamber 15 can be changed. The temperature in the culturing chamber 15 can be monitored by the controller 16 by means of the temperature sensor 10 shown, which is connected via a data line 30 to the controller 16. Furthermore, the controller 16 can also exert a controlling action on the heating element 28 via the data line 30 shown (by specifying an electrical heating current) and thus set a heating power as a further action parameter 3b, which can be supplied to the culturing chamber 15 and thus the biological process 2 by means of the heating element 28.

The apparatus 1 further comprises a camera 19 as a measuring means 17, with which microscopic images of the biological sample, in particular the biological system 34, such as cells of a cell culture 33 that grow in the culturing chamber 15 can be recorded.

Via the drainage line 25, the culture medium 13 can be discharged at regular intervals and transported to the measuring point 20 shown. A further measuring means 17 in the form of a spectrometer 18 is connected at the measuring point 20 via a glass fiber 32 and can thus spectroscopically measure the culture medium 13 at the measuring point 20. A microfluidically active separating structure 11 in the form of a filter ensures that the elements of the biological system 34, which are cultured in the culturing chamber 15, are kept distant from the measuring point 20 so that the spectrometric measurement carried out with the spectrometer 18 takes place under exclusion of the biological system 34, wherein for this purpose only the culture medium 13, but not the elements 14 contained therein of the biological system 34, are illuminated. In other applications, the filter can also be removed so that the spectrometer 18 then conducts spectral measurements of both the culture medium 13 and the elements 14 contained therein. In this manner, for example, an OD600 measurement could be carried out (see further above).

After the culture medium 13 has passed the measuring point 20, it flows via the further drainage line 25 into a collection container 22.

As indicated by the dotted line in FIG. 1, a part of the apparatus 1 is configured as a microfluidic device 21. This serves on the one hand to supply the biological system 34 with the culture medium 13; on the other hand, to record microscopic images with the camera 19 and finally the spectrometric measurement explained above. For this purpose, the glass fiber 32 is integrated into the microfluidic device 21, while the actual measurement assembly of the spectrometer 18 is arranged outside a housing 31 of the apparatus 1 that accommodates the other components of the apparatus 1.

The spectrometer 18 is also controlled and read by the controller 16. By means of the spectrometric measurement, it is also possible to carry out indirect acquisition of the chemical substances or biological organisms produced by the biological process 2 (which is known as chemo- or biogenesis).

By means of the spectrometric measurement, it is therefore possible to carry out indirect acquisition of both a consumption of specified substances and the production of specified substances by the biological process 2. This acquisition takes place precisely during the biological process 2 and while—by setting of the action parameters 3—the biological process 2 is being influenced.

The controller 16 of the apparatus 1 is now configured for setting the two action parameters 3a, 3b, i.e. the flow rate 3a at which the substance 26 reaches the culturing chamber 15 and the heating power 3b that is supplied by means of the heating element 28 to the culturing chamber 15 and thus the biological process 2. By means of setting the action parameters 3, the controller 16 influences conditions that influence the biological process 2, which allows the biological process 2 to be controlled.

The controller 16 (which can be a microcontroller) further comprises a memory 9, in which a preset target value 5 is stored in the form of a target spectrum.

In reaction to the action parameters 3a, 3b set by the controller 16, the biological process 2 changes, thus causing a metabolic activity of the microorganisms 14 to change. On the one hand, this leads to an increase in metabolic consumption; on the other hand, however, the microorganisms 14 also produce metabolic products, which they release into the culture medium 13. In other words, the biological process 2, or the biological system 34 responsible for this process, depending on the action parameters 3a, 3b set, responds with a particular process response 4, which the biological process 2 transfers in reaction to an adjustment of at least one of the two action parameters 3a, 3b to its immediate environment, i.e. the culture medium 13.

On the one hand, this process response 4 is spectrometrically measured by the controller 16 by means of the spectrometer 18, wherein the spectrum measured at the measuring point 20 of the culture medium 13 is stored as a measurement parameter 8a of the process response 4 in an internal memory 9 of the controller 16.

By means of the temperature sensor 10, the controller 16 can further measure a temperature increase of the biological sample 12, which is formed in the culturing chamber 15 by the culture medium 13 and the biological system 34, as a second measurement parameter 8b of the process response 4. This temperature increase 8b is also stored in the memory 9 as a further measurement parameter 8 and thus as a further component of the process response 4. Here, the temperature increase is measured at points in time when no heating power is being supplied, so that the temperature increase 8b can be fed back to the biological process 2, i.e. is produced thereby (thermogenesis). This second measurement parameter 8b is also stored in the memory 9 and can thus be further processed by the controller 16.

Moreover, by means of the biological process 2, which is fueled by the biological system 34 and takes place in the culturing chamber 15, numerous environmental factors are changed, i.e. for example the material composition of the culture medium 13, its temperature, its pH, the electrical conductivity of the culture medium 13 and also its optical transmission behavior. In general, all of these environmental factors can be measured according to the method according to the invention as individual measurement parameters 8 of the process response 4 of the biological process 2 in order in this manner to acquire respective changes in these environmental factors in reaction to the respective adjustment of one of the action parameters 3.

In a following step, the controller 16 compares the two measured measurement parameters 8a, 8b with a respective preset target value 5, i.e. in one case a target spectrum 5a (as a first target variable) and in one case a target value for the temperature increase 5b (as a second target variable). After this, each of the two measurement parameters 8a and 8b acquired as a process response 4 is evaluated by the controller 16 in a computer-implemented manner based on the respective preset target value 5a, 5b by means of a respective evaluation 6a, 6b. The controller 16 then adjusts either the action parameter 3a or the action parameter 3b, or however both action parameters 3a, 3b in a computer-implemented manner based on these two evaluations 6a, 6b.

This adjustment of the action parameters 3a, 3b takes place with the goal of bringing the measured process response 4 closer to the preset target values stored in the memory 9 as reference responses, i.e. with the goal of controlling the biological process 2 in such a way that a desired temperature increase and a specified spectrum are obtained as a process response 4. In contrast to the regulation of the overall temperature in the culturing chamber 13 by means of the heating element 28, in this case, for example, there is no measurement of how a supplied heating power changes the temperature in the culturing chamber 15; rather, the temperature sensor 10 is used to acquire the amount of heat produced by the metabolic activity of the biological system 34 as a part of the process response 4, which is known by the term thermogenesis.

It is obvious that this approach can for example also be expanded to the detection of a photogenesis, i.e. the production of light by a metabolic activity of the biological system 34, wherein the light produced by the biological process 2 would then be measured by means of a photosensitive sensor. This also differs for example from the measurement of a light intensity that is supplied by means of a light source—for example as a further possible action parameter 3—to the culturing chamber 15, for example in order to induce photosynthesis in said chamber by means of microorganisms 14.

The controller 16, which can be configured as a microcontroller, learns the adjustment to be carried out of set values of the two action parameters 3b and 3c by means of an artificial intelligence, which is implemented in the form of an artificial neural network (ANN). In other words, the adjustment of the action parameter 3 and the evaluation of the respective measured measurement parameters 8a, 8b are based on a machine learning method.

The machine learning makes it possible for the controller 16 to learn independently and in a computer-implemented manner, based on multiple evaluations 6, how the individual action parameters 3 are set such that the measured process response 4 is brought closer and closer to a desired process response 4, which describes a status of the biological process 2 that is to be achieved by control by means of the apparatus 1.

It thus becomes clear from these explanations that the controller 16, by means of the temperature sensor 10 and the spectrometer 18, measures two measurement parameters 8, i.e. the measured spectrum 8a and an endogenous temperature increase 8b within the biological sample 12, then evaluates the process response 4 composed of these two measurement parameters 8a, 8b by means of a respective evaluation 6a, 6b in a computer-implemented manner, and subsequently adjusts the respective set values of the action parameters 3a, 3b in a computer-implemented manner based on the evaluation 6.

The adjustment of the action parameters 3a now takes place in that on the one hand, the controller 16, by controlling the valve 29, controls the amount of material 26 that is removed from the reservoir 27 and fed to the biological process 2 in the culture medium 13. On the other hand, the controller 16 can set the action parameter 3b, i.e. the heating power, by sending a larger or smaller amount of control current to the heating element 28, so that the element supplies more or less heat to the culturing chamber 15. This means that the controller 16 can use the temperature sensor 10 once as a measuring means 17 in order to measure the measurement parameter 8b of the thermogenesis generated by the biological process 2. On the other hand, however, the controller 16 can also use the temperature sensor 10 to regulate the heating power 3b generated by means of the heating element 28. In particular, it is obvious that the temperature sensor 10 can be used alternatingly for these two tasks, for example at predetermined time intervals of the process control.

As has already been discussed, the evaluation of the process response 4 is carried out on the basis of respective preset target values 5a, 5b for the individual measurement parameters 8 of the process response 4. The two measurement parameters 8a and 8b discussed so far, i.e. the measured spectrum 8a and the endogenous temperature increase 8b in the biological sample 12, can be understood in each case as overall measurement parameters, as they are influenced by all of the microorganisms 14 contained in the culture medium 13. Here, at least the spectrum 8a is acquired as an overall measurement parameter without a direct measurement of the microorganisms 14.

By means of the camera 19 shown, a further measurement parameter 8 can also be acquired. This parameter can in particular also be a measurement parameter 8 of a status of the biological process such as the number of cells 8c that can be acquired within a predetermined viewing window of the microfluidic device 21 by means of an image processing of the camera 19 at a specified point in time. In a variant, moreover, the invention precisely now proposes that such a measurement parameter be evaluated via a process status on the basis of a preset target value 5 and that the adjustment, based on this evaluation, then be carried out by at least one of the above-discussed action parameters 3.

Moreover, the parameter 8c can be understood as a local parameter, as it is only influenceable by individual elements of the biological system 34. For example, the increase in microorganisms 14 outside of the viewing window has no influence whatsoever on the measurement of the number 8c of cells within the viewing window. This local measurement parameter 8c further differs from the two other measurement parameters 8a and 8b in that it is acquired by means of a direct measurement of individual elements of the biological system 34, i.e. by an image processing of microscopic images of these elements recorded with the camera 19.

As the measured spectrum and the microscopic images are simultaneously acquired and thus describe a current status of the biological process 2 in each case, the measured spectrum can be used as a first measurement parameter 8a of the acquired process response 4 to correspondingly adjust an evaluation criterion that is used to generate an evaluation 6c of the number of cells per microscopic image, which is used as a further measurement parameter 8c, such that a meaningful evaluation of the recorded microscopic images can take place. In other words, it is thus proposed, in particular by using a method of self-supervised learning, that the recorded spectrum 8a be considered a basic truth, and that the learned evaluation 6a of the spectrum (which takes place using the target spectrum stored in the memory 9) be used in order to improve the evaluation 6c of the number 8c of cells in the individual microscopic images. To put it simply, the apparatus thus independently learns the number of cells in a specified status of the biological process 2 that can reasonably be expected or are achievable, specifically based on the learned evaluation of the measured spectrum 8a, which allows a conclusion to be drawn with respect to the current status of the biological system 34/of the biological process 2.

Conversely, however, the microscopic images, more specifically the measurement parameter of a specified cell density derived therefrom, can also be used to train an AI to correctly (i.e. meaningfully) evaluate recorded spectra 8a. Generally speaking, it can thus be provided in particular that a measurement parameter which describes a status of the biological process 2 is used by an, in particular the above-mentioned, AI to learn an evaluation 6a of a measurement parameter 8a of the process response 4.

In summary, it is provided that, for improved autonomous control of a process 2 by means of an apparatus 1 by setting at least one action parameter 3 that controls the process 2, using a measuring means 17, preferably a spectrometer 18 integrated into the apparatus 1, a process response 4 which the process 2 transfers to its immediate environment in reaction to an adjustment of the at least one action parameter 3 is measured and evaluated in a computer-implemented manner, preferably using an artificial intelligence, and that based on this evaluation, the at least one action parameter 3 is automatically readjusted by the apparatus 1, for example in order to guide the process 2 in a desired direction. This approach is applicable to biological, chemical and physical processes.

### LIST OF REFERENCE NUMBERS

1 Apparatus (e.g. configured as an incubator)
2 Biological/chemical/physical process (which takes place in 1, more precisely in 15)
3 Action parameter (for controlling 2)
4 Process response (of 2)
5 Preset target value (target)
6 Evaluation (of 4)
7 Control variable (for influencing 3)
8 Measurement parameter (as a part of 4)
9 Memory
10 Temperature sensor
11 Separating structure (e.g. membrane, filter, through-flow opening)
12 Sample (particularly biological sample)
13 Medium, in particular culture medium
14 Elements of 34 (e.g. cells, microorganisms, bacteria, algae, etc.)
15 Process chamber (particularly culturing chamber for culturing a biological sample)
16 Controller
17 Measuring means
18 Spectrometer
19 Camera
20 Measuring point
21 Microfluidic device (e.g. microfluidic chip)
22 Collection container
23 Actuator
24 Supply line
25 Drainage line
26 Material
27 Reservoir (for 26)
28 Heating element
29 Valve
30 Data line
31 Housing
32 Glass fiber
33 Cell culture
34 System (particularly biological system)

1. A method for controlling an apparatus (1), in which a process (2) takes place that is carried out by a biological system (34) or a non-biological system, and the process (2) is controlled by setting at least one action parameter (3), the method comprising:
measuring a process response (4) of the process (2) on the at least one action parameter (3);
evaluating the process response (4) with a computer-implemented evaluation (6) using a preset target value (5); and
adjusting at least one set value of the at least one action parameter (3) in a computer-implemented manner based on the evaluation (6).

2. The method as claimed in claim 1, wherein at least one of:
the predetermined target value (5) is a reference response (5) for the process response (4), and the adjusting is for aligning the process response (4) with the predetermined target value (5); or
process (2) takes place in a sample (12) that comprises a culture medium (13) and the process response (4) is measured in the culture medium (13).

3. The method as claimed in claim 1, further comprising measuring the process response (4) of the process (2) on a medium (13) that surrounds elements (14) of the system (34) on which the process (2) is based, and the system (34) is embedded in the medium (13) which forms a culture medium (13).

4. The method as claimed in claim 3, further comprising measuring at least one measurement parameter (8) of the process response (4) in an immediate environment of the elements (14) of the biological system (34) on which the process (2) is based, without direct measurement of the elements (14) of the biological system (34).

5. The method as claimed in claim 1, wherein by measuring the process response (4), at least one of a consumption or a production of at least one material is acquired by the process (2), at least indirectly, and wherein at least one of:
the process (2) is influenced by setting the at least one action parameter (3); or
by measuring the process response (4), an activity of the system (34) including at least one of (i) a thermogenesis (production of heat by metabolic activity), (ii) a chemo- or biogenesis (production of chemical substances or biological organisms by metabolic activity), (iii) a photogenesis (production of light by metabolic activity), an energy consumption, or (iv) a material consumption or a material production, is acquired.

6. The method as claimed in claim 1, wherein the process (2) changes at least one environmental factor, including at least one of a material composition, a temperature, a pH, a permittivity, an electrical conductivity, an optical transmission or reflection behavior, or an environmental factor of a culture medium (13) in which the biological system (34) is cultured, and the method further comprises measuring the at least one environmental factor as at least one measurement parameter (8) of the process response (4).

7. The method as claimed in claim 1, further comprising acquiring at least one measurement parameter (8a) of the process response (4) using a spectrometric measurement.

8. The method as claimed claim 1, wherein the adjusting of the respective set value of the at least one action parameter (3) is computer-implemented via an artificial intelligence (AI).

9. The method as claimed in the claim 8, wherein the AI uses data sets comprising
a respective measured process response (4) and
an accompanying set of action parameter values producing said process response (4)
in order to at least one of prepare or optimize a prediction model for the system response (4), and
the AI, with the help of the prediction model, generating virtual system responses in reaction to respective virtual sets of action parameter values, and validating said virtual system responses using real tests.

10. The method as claimed in claim 1, wherein at least one optimized set value for the at least one action parameter (3) is independently learned in a computer-implemented manner by the apparatus (1) based on several evaluations (6) derived by the apparatus (1) from respective ones of the process responses (4), in each case in reaction to set values of the at least one action parameter (3) specified by the apparatus (1).

11. The method as claimed in claim 1, wherein in addition to the process response (4), the method further comprises measuring a process status, and a first measurement parameter (8) is measured by the process response (4) and a second measurement parameter (8) is measured by the process status.

12. The method as claimed in claim 1, further comprising measuring at least two measurement parameters (8) and using one of the two measurement parameters (8) in order to verify an evaluation carried out using the other measurement parameter (8).

13. The method as claimed in claim 1, further comprising measuring at least two measurement parameters (8), and the measurement parameters (8a, 8b, 8c) are selected from the following group of measurement parameters (8):
optical measurement variables including at least one of an absorption spectrum, an emitted light intensity, or a fluorescence;
electrical variables, including at least one of an electrical conductivity or permeability;
thermal variables, including a self-heating of a biological sample; or
pH values.

14. The method as claimed claim 13, further comprising evaluating each of the measurement parameters (8), using a respective preset target value (5a, 5b) using a respective computer-implemented evaluation (6a, 6b), and
adjusting at least one set value of the at least one action parameter (3) in a computer-implemented manner based on the evaluations (6a, 6b).

15. The method as claimed in claim 1, further comprising using a first measurement parameter (8a) to at least one of verify or adjust an evaluation criterion which is used to generate an evaluation (6b) of a second measurement parameter (8b), via a method of self-supervised learning, in which the first measurement parameter (8a) is taken as a basic truth in order to improve the evaluation of the second measurement parameter (8b).

16. The method as claimed in claim 15, wherein at least one of the measurement parameters (8a, 8b, 8c) is an overall measurement parameter (8) that is influenceable by all elements (14) of the biological system (34), and at least one of
(a) the overall measurement parameter (8) is acquired without direct measurement of the elements (14) of the biological system (34),
or
(b) at least one of the acquired measurement parameters (8a, 8b, 8c) is a local measurement parameter that is only influenceable by individual ones of the elements (14) of the biological system (34), and the local measurement parameter is acquired by direct measurement of the individual elements (14) of the biological system (34).

17. An apparatus (1) for the autonomous control of a process (2), comprising:
a process chamber (15) for accommodating a biological, chemical or physical system, in which the process (2) takes place,
a controller (16) configured to set at least one action parameter (3) in order to control the process (2) using the action parameter (3), and
at least one measuring device (17) for measuring a process response (4) of the process (2), which takes place in reaction to an adjustment of the at least one action parameter (3).

18. The apparatus (1) as claimed in the claim 17, wherein the apparatus is configured to control a biological process (2), and at least one of
(a) the at least one of the measuring device (17) is configured to acquire an overall measurement parameter (8) of the process response (4), which is changeable or is changed by all elements (14) of the biological system (34) on which the process (2) is based, or
(b) the at least one of the measuring device (17) is configured to acquire the overall measurement parameter (8) and is arranged such that the measurement of the process response (4) takes place at least partially at a measuring point (20) that is kept free from the elements (14) of the biological system (34).

19. The apparatus (1) as claimed in claim 18, further comprising a microfluidic device (21) for supplying the system (34) with a medium, and at least one of
(a) the microfluidic device (21) comprises a microfluidically active separating structure (22) with which the elements (14) of the biological system (34) are maintained at a distance from the measuring point (20) at which the process response (4) is acquired via the at least one measuring device (17), or
(b) the at least one measuring means (17) is at least partially integrated into the microfluidic device (21) and the measuring point (20) is therefore unchangeable with respect to the separating structure (22).

20. The apparatus (1) as claimed in claim 17, further comprising at least one actuator (23) configured to change the at least one action parameter (3), and
the at least one actuator (23) is regulatable by the controller (16),
the controller (16) is configured to adjust at least one set value of the at least one action parameter (3) in a computer-implemented manner by an evaluation (6) of the process response (4) in order to align the process response (4) with a preset target value (5).
